(19) United States
(12) Patent Application Publication
(10) Pub. No.: US 2021/0060377 A1
(43) Pub. Date: Mar. 4, 2021

(54) BAND RESISTANCE STRENGTH PLATFORM

(71) Applicant: Maitrick's Design, LLC, Chagrin Falls, OH (US)

(72) Inventor: Zoltan Mesko, Chagrin Falls, OH (US)

(73) Assignee: Maitrick's Design, LLC, Chagrin Falls, OH (US)

(21) Appl. No.: 17/003,410

(22) Filed: Aug. 26, 2020

Related U.S. Application Data
(60) Provisional application No. 62/891,655, filed on Aug. 26, 2019.

(51) Int. Cl. A63B 21/00; A63B 21/04; A63B 21/055; A63B 24/00

(52) U.S. Cl. CPC A63B 21/4033; A63B 21/0442; A63B 21/0557; A61B 5/4023; A63B 2220/50; A63B 2220/833; A63B 24/0062

(57) ABSTRACT

An exercise apparatus includes a platform. The platform includes a first side and a second side. The first side bears a user. The second side includes one or more rollers. A resistance band wraps around the second side and passes over the rollers. The resistance band extends towards a user on the first side.

### CROSS-REFERENCE TO RELATED APPLICATIONS

This application claims priority to U.S. Provisional Patent Application No. 62/891,655 filed on Aug. 26, 2019, entitled “BAND RESISTANCE STRENGTH PLATFORM,” the entirety of which is incorporated herein by reference.

### TECHNICAL FIELD

The present invention relates to exercise equipment apparatuses, and more particularly to exercise equipment apparatuses that simulate the hex or trap bar deadlift through resistance.

### BACKGROUND

The hex or trap bar deadlift has become a widely adopted lift in commercial and home gyms across the globe. It has gained popularity from elite professional athletes to casual lifters due to the exercise being capable of activating the majority (approximately 80%) of a person's skeletal musculature. This allows a person to get a comprehensive workout through performing just one exercise. Other Olympic or compound lifts such as the squat, conventional or straight bar deadlift, power clean, hang clean, and other versions of lifts do accomplish a similar level of activation, however, such exercises require a lifter be highly skilled and refined in their technique in order to avoid injury.

Traditionally, hex or trap bar exercises require weights, machines, or the like. Such weights and machines pose size, expense, and convenience related problems with the hex or trap bar that lies in it being a heavy and bulky item that requires a wide range of free weights to accompany it, therefore making the whole set inconvenient for a person to have in their own home. Because of the inability to have an all-encompassing weight set, squat rack, or other bulky free weight based weight system at home, busy individuals may also not make exercising their lower bodies a priority and will skip out on going to the gym due to time constraints. Many people can exercise their upper bodies at home with pull-ups via various door frame mounted pull-up machines, push-ups performed on the ground, and utilizing various dumbbell, kettlebell, and resistance band exercises. Lower body exercises with a complete range of resistance, however, are impossible to replicate at home without the heavy and bulky free weight based equipment.

Compared to other compound movements in resistance training such as the squat and conventional or straight bar deadlift, the hex/trap bar deadlift is relatively safe to use. Due to its design, it allows a user to lift a load that is located close to the center of a person's mass. The lift is performed with a neutral grip, which minimizes the risk of tearing one's arm muscles or connective tissues. It also places relatively less stress on one's hamstrings and lower back as compared to other Olympic or compound lifts. However, because the amount of resistance loaded does not change with the user's position during the lift, the bottom portion of the lift does place a user's lower back into a vulnerable position given relatively heavy weight

The field of exercise has seen various sizes and shapes of resistance bands come to market, which allows users to supplement their free weight based resistance training. The issue is that those resistance bands utilized individually to perform the equivalent of a hex or trap bar deadlift either do not provide enough resistance, or become unsafe to use when multiple bands are stepped onto to be pulled up from the ground by the lifter.

### SUMMARY

The following presents a summary of this disclosure to provide a basic understanding of some aspects. This summary is intended to neither identify key or critical elements nor define any limitations of embodiments or claims. Furthermore, this summary may provide a simplified overview of some aspects that may be described in greater detail in other portions of this disclosure.

Described herein is an exercise assembly comprising: a platform comprising a first side and a second side, the first side operatively bearing a user; at least one roller device disposed beneath the second side when viewed from the first side; and at least one cutout formed through the platform from the first side to the second side, the at least one cutout operatively disposed to provide line of sight with the at least one roller when viewed from the first side, such that a resistive band may pass through the at least one cutout and pass over the at least one roller device, and wherein terminal ends of the resistive band extend above the first side. The exercise assembly may comprise at least one support extending from the second said. The at least one roller may be disposed between two of the at least one support. The platform may comprise a first edge and a second edge, and wherein the at least one support comprises four supports disposed at general corners of the second side of the platform, and wherein the at least one roller comprises a first roller disposed between two of the supports proximal the first edge and a second roller disposed between the two other supports proximal the second edge. The at least one roller may comprise at least one of a dowel rod, an axle comprising one or more bearings, an axle mounted with or on bearings, an axle with one or more bearing beads circumventing the axle, or ball bearing surfaces. The platform may comprise a first edge and a second edge opposite the first edge. The one or more cutouts may comprise a first cut out proximal the first edge and a second cutout proximal the second edge. The one or more cutouts may comprise a first cut out and a second cutout wherein the first cutout is positioned a distance from the first edge and the second cutout is positioned the distance from the second edge. The one or more cutouts may comprise a cutout positioned in the general center of the platform. The exercise assembly may comprise an anti-slip surface disposed on the first side.

Also described is an exercise assembly comprising a platform comprising a first side and a second side, a first edge, and a second edge, the first side operatively bearing a user, the second side opposed to the first side, and the first edge opposed to the second edge; a plurality of supports extending from the second side; at least one roller device disposed beneath the second side when viewed from the first side, and rotatably attached to the plurality of supports; and at least one cutout formed through the platform from the first side to the second side, the at least one cutout operatively disposed to provide line of sight with the at least one roller when viewed from the first side, such that a resistive band may pass through the at least one cutout and pass over the at least one roller device, and wherein terminal ends of the resistive band extend above the first side. The exercise assembly may comprise at least one handle operatively attaching to terminal ends of the resistive band from the first side. Forcing the at least one handle away from the first side operatively stretches the resistive bands, and wherein the at least one roller device operatively allows the resistive band to deform such that resistance on terminal ends of the resistive bands are generally equalized. The at least one handle may comprise gripping portion operatively providing a gripping surface for a user and at least one hooking portion operatively attaching to the terminal ends of the resistive band. The at least one hooking portion may comprise a planar surface for receiving the resistive band. The at least one hooking portion may comprise at least one roller device for receiving the resistive band. The gripping portion may comprise an ergonomic handle.

An exercise assembly may comprise a platform comprising a first side and a second side, a first edge, and a second edge, the first side operatively bearing a user, the second side opposed to the first side, and the first edge opposed to the second edge, and further comprising at least one sensor disposed on or within the platform for sensing pressure operatively applied by a user during exercise; a plurality of supports extending from the second side; at least one roller device disposed beneath the second side when viewed from the first side, and rotatably attached to the plurality of supports; and at least one cutout formed through the platform from the first side to the second side, the at least one cutout operatively disposed to provide line of sight with the at least one roller when viewed from the first side, such that a resistive band may pass through the at least one cutout and pass over the at least one roller device, and wherein terminal ends of the resistive band extend above the first side. The at least one sensor is operatively coupled to a controller that determines an amount of resistance during an exercise. The controller further determines a balance metric associated with the user during the exercise.

The following description and the drawings disclose various illustrative aspects. Some improvements and novel aspects may be expressly identified, while others may be apparent from the description and drawings.

### DESCRIPTION OF THE DRAWINGS

The present teachings may be better understood by reference to the following detailed description taken in connection with the following illustrations, wherein:

FIG. 1 is a top perspective view of a platform in accordance with the present disclosure;

FIG. 2 is a perspective view of a handle system in accordance with the present disclosure;

FIG. 3 is an enlarged view of the embodiment of FIG. 1 illustrating a bearing system in accordance with the present disclosure;

FIG. 4 is an embodiment of the platform of FIG. 1 and handles with resistance bands in accordance with the present disclosure;

FIG. 5 is an embodiment of the platform of FIG. 1 without handles in accordance with the present disclosure;

FIG. 6 is an enlarged view of a ball bearing and roller assembly in accordance with the present disclosure;

FIG. 7 illustrates an example exercises simulating a seated row with the platform of FIG. 1 in accordance with the present disclosure;

FIG. 8 illustrates an example exercises simulating hex bar deadlifts with the platform of FIG. 1 in accordance with the present disclosure;

FIG. 9 illustrates is a top perspective view of another platform with additional rollers than that shown in FIG. 1, in accordance with the present disclosure;

FIG. 10 is a bottom view of the platform of FIG. 9 in accordance with the present disclosure;

FIG. 11 is a platform with a straight bar in accordance with the present disclosure;

FIG. 12 is a smart platform in accordance with the present disclosure;

FIG. 13 is an example network for the smart platform of FIG. 18 in accordance with the present disclosure; and

FIG. 14 is an example operating environment for the smart platform of FIG. 18 in accordance with the present disclosure.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION

Reference will now be made in detail to embodiments of the present teachings, examples of which are illustrated in the accompanying drawings. It is to be understood that other embodiments may be utilized and structural and functional changes may be made without departing from the scope of the present teachings. Moreover, features of the embodiments may be combined, switched, or altered without departing from the scope of the present teachings, e.g., features of each disclosed embodiment may be combined, switched, or replaced with features of the other disclosed embodiments. As such, the following description is presented by way of illustration and does not limit the various alternatives and modifications that may be made to the illustrated embodiments and still be within the spirit and scope of the present teachings.

As used herein, the words “example” and “exemplary” mean an instance, or illustration. The words “example” or “exemplary” do not indicate a key or preferred aspect or embodiment. The word “or” is intended to be inclusive rather than exclusive, unless context suggests otherwise. As an example, the phrase “A employs B or C,” includes any inclusive permutation (e.g., A employs B; A employs C; or A employs both B and C). As another matter, the articles “a” and “an” are generally intended to mean “one or more” unless context suggests otherwise.

In described embodiments, an exercise apparatus comprises a platform. At least one roller is disposed on a second (e.g., bottom) side of the platform. A first side of the platform may comprise a surface which a user may stand on, sit on, or otherwise interact with. The platform may include at least one leg disposed on the second side. The roller may be mounted between a plurality of legs. In at least one example, legs may include bearings, such as ball bearings. The roller may be disposed in or mounted with the bearings. One or more resistance bands, such as elastomeric bands, may pass over the rollers on the second side and be held by a user on the first side. As such, the one or more resistance bands may form a u-shape around the platform with a user holding either end of the one or more resistance bands.

In at least some examples, the exercise apparatus may include one or more gripping devices, such as a handle, straight bar, curl bar, or the like. In an aspect, the gripping device may receive and attach to the one or more resistance bands. For instance, the user may place one or more resistance bands under the platform on which the user stands on, such that the one or more resistance bands pass over and contacts one or more rollers. The resistance bands may then come up from underneath the platform, along the left and right sides of the user. As such, the resistance bands travel along the one or more rollers, such as two rollers placed on opposite ends of the first side of the platform. The rollers eliminate or reduce friction that would be caused by one or more bands contacting an edge of the platform or other surface. In another aspect, by allowing each resistance band to extend from two sides, the resistance on each end of the band is equalized. For instance, in some traditional systems, multiple bands may be required where a band extends from only one side of a device, and this may cause imbalances as bands wear or lose elasticity at different rates or are otherwise unbalanced.

According to at least one example, the exercise apparatus may include two handles that hold onto the bands coming upward from the platform. The handles allow the user to conveniently pull one or multiple bands at a time, which provides a progressively increasing resistance felt as the user moves the handles away from the platform during exercise. This system allows for a user to simulate a resistance at the top of the lift from only a few pounds to multiple hundreds of pounds, all while starting with zero resistance at the bottom. The bottom portion of the lift being zero to very little resistance is important as the bottom portion of the hex or trap bar deadlift is mechanically the most compromising position for a lifter's spine and lower back.

In some examples, a handle may comprise a gripping portion and a hooking portion. The gripping portion may comprise an ergonomic grip which a user may grasp with their hand. The hooking portion may receive one or more resistance bands. The handle may be shaped such that the gripping portion is generally coaxial with a center of mass of the hooking portion. In some instances, the hooking portion may comprise an opening to allow for quick detachment of one or more resistance bands. In other instances the hooking portion may comprise a latch or clasp that may be opened and closed to secure one or more bands therein.

The present disclosure provides a solution to the problem by providing a band resistance strength platform. As described herein, disclosed embodiments utilize one or more rollers to equalize the resistance or force on either side of a resistance band. As described herein, a roller can include a dowel rod, an axle comprising one or more bearings, an axle mounted with or on bearings, an axle with one or more bearing beads circumventing the axle, ball bearing surfaces, or the like.

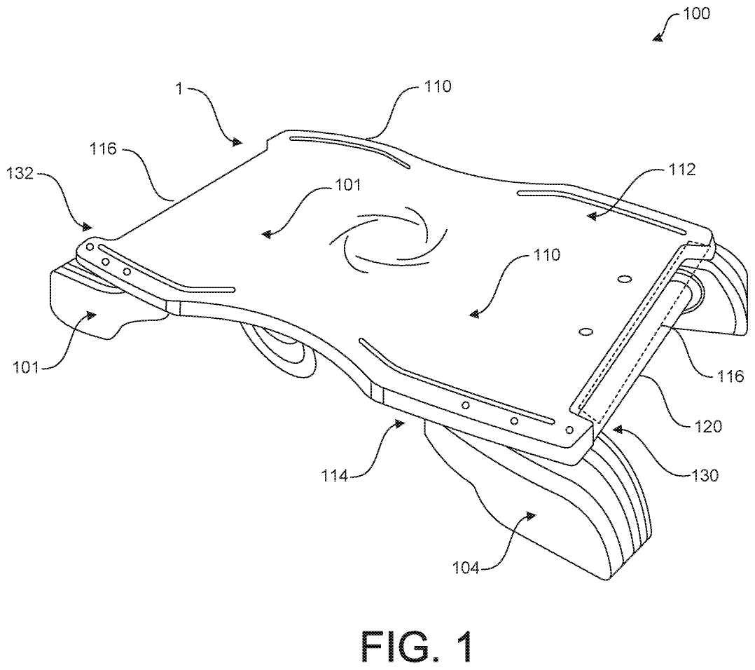

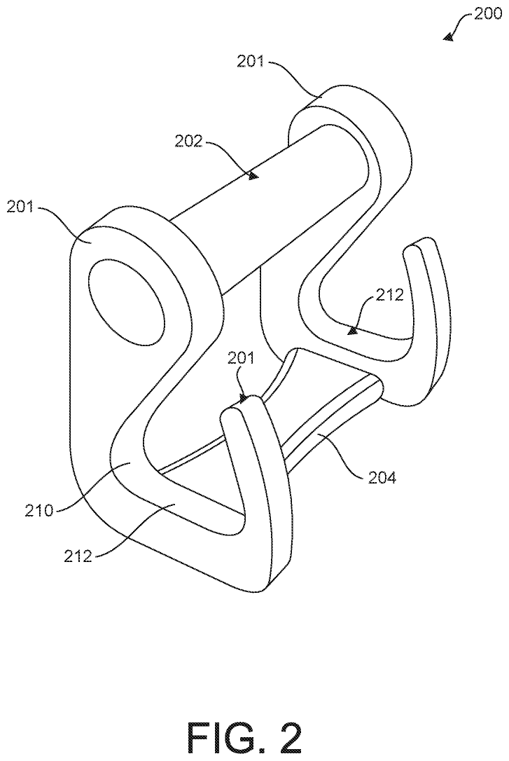

In FIG. 1, there is a system 100 comprising a platform 110 and at least one roller 120. The platform 110 can be made out of any sturdy material or combination of materials such as wood, plastic, metal or any other material that would not buckle under the weight of a person. It is noted that the system 100 may be utilized with other systems, such as a handle system 200 as depicted in FIG. 2.

The platform 110 may comprise a first side 112 and a second side 114 opposed to the first side 112. The first side 112 may generally provide a surface on which a user may stand or otherwise apply pressure. In at least some embodiments, the first side 112 may comprise anti-slip surface 101 coatings, formations, or the like that provide for additional friction (e.g., friction adhesives, tapes, treads, magnets, etc.). In another aspect, the first side 112 may comprise indicia indicating foot placements, exercise instructions, or the like.

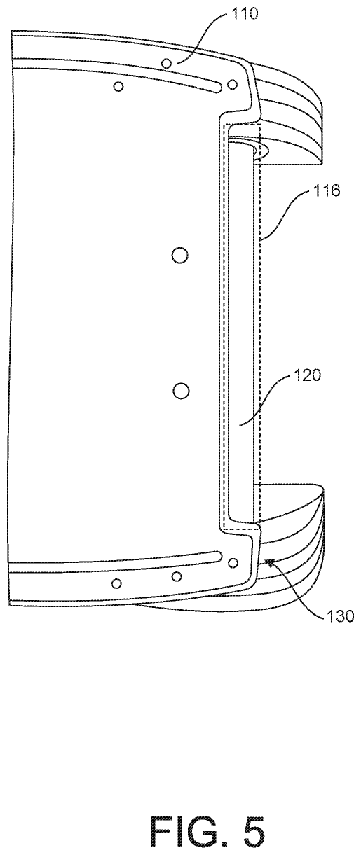

In at least some embodiments, the platform 110 may include at least one cutout 116. The cutout 116 may be disposed along one or more edges of the platform 110, such as first edge 130 and second edge 132. The cutout 116 allows space for a resistance band in direct line of sight with the rollers 120 which may be disposed proximal, below, or extending from the second side 114 of the platform 110. For instance, the cut out 116 may comprise an area or aperture internal to the platform 110 relative first edge 130 and second edge 132. This may allow the platform 110 to extend beyond the roller 120 when viewed from first side 112 to provide additional stability while reducing wear on a resistance band, such as shown in FIG. 5. In another aspect, the placement of the roller 120 away from an edge may reduce accidental slippage from a user contacting the roller 120 during use. It is noted that cutouts 116 may be disposed along any edge, centered on edges, or at other locations.

In various examples, the platform 110 may include cutouts 116 disposed within the platform 110 as apertures or slits away from an edge. A user may pass a resistance band through an appropriate slit, such that the user may select a desired width. It is noted that such examples may include adjustably positioned rollers or multiple rollers corresponding to the different slits. This may allow a user to select a width where resistance is generally pulled downward rather than at a non-90 degree angle. In various other examples, the platform 110 may not comprise cutouts 116. In such examples, the rollers 120 may be disposed to extend beyond the terminal edge of the platform 110 to allow a resistance band to be free of contact.

As described here and elsewhere in this disclosure, rollers 120 may be positioned such that resistance bands interact or contact the rollers 120 during operation. Generally, the rollers 120 do not contact any other portion of the system 100. The rollers 120 minimize friction as the resistance bands are pulled and stretched under tension. The rollers 120 also allow the resistance band portion running underneath the platform to be stretched, therefore optimizing the length that one or more resistance bands can be stretched to. This is important as the lifter will experience a more gradual increase in tension or resistance felt beginning at zero resistance to the maximal allowable resistance a particular band is designed for. The rollers 120 also allow for the band to uniformly stretch to either side, having the user experience an equilibrium in the resistance felt on either side. In traditional systems, resistance bands may be fixed to an anchor point, clamped, or held in place by the weight of a user. As such, traditional systems do not balance the forces or resistance throughout the band. It is further noted that terminal edges of the platform 110 may include beveled, angled, curved, or otherwise non-sharp edges such that friction and wear on bands may be further reduced in the event that a band does contact a terminal edge of the platform 110.

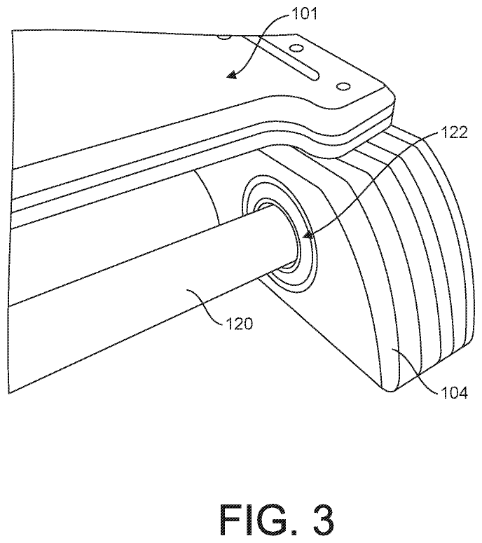

Rollers 120 may comprise various constructions as described herein. As shown, rollers 120 comprise an axel mounted with ball bearings 122. As magnified in FIG. 3, the ball bearings 122 are housed in one or more legs or supports 104 of the platform 110. In various examples, the legs or supports 104 may extend beyond the platform 110 to support or hold the rollers 120 and ball bearings 122 without the support of the platform 110 being positioned on top of the legs or supports 104.

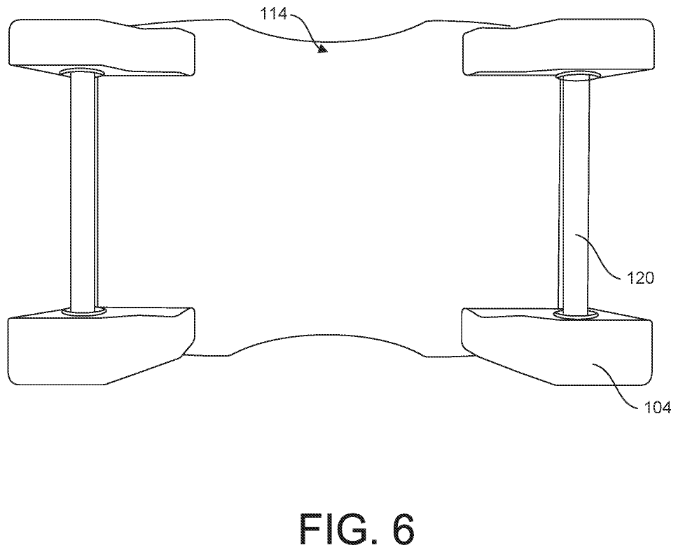

With reference to FIG. 6, the supports 104 may be positioned to extend from second side 114. It is noted that i supports may be provided, where i is a number. As shown in FIG. 6, four supports 104 may be provided in general corners to allow for stability of the system 100. Moreover, two rollers 120 may be provided and may be disposed between supports 104. These supports or legs 104 allow for the proper clearance for one or multiple bands to be utilized underneath the platform 110 and may contact a surface, such as a floor. It is noted that platform 110 may include any number of supports 104. In another aspect, some supports 104 may not extend to a floor, may not include ball bearings, and may comprise different constrictions than what is illustrated.

It is further noted that embodiments may utilize other configurations of rollers 120 that turn near frictionlessly. For instance, a plurality of ball bearings or wheels may be mounted on one or more axles. The axle may be held in place while the ball bearings may be rotatable about the axle. In another example, the rollers may comprise an outer tube that may be disposed about a stationary central axle or dowel rod such that the bands may travel along the rotating piece and avoid friction of the resistance bands at the edges of the platform. As also described elsewhere in this disclosure, bearing assemblies may include roller beads mounted on an axle, belt and roller systems (e.g., similar to treadmills), ball bearings, or the like.

The rollers 120 could be made out of wood, metal, plastic, but is not limited to and could be any material able to withstand any small or large amount of force placed upon it. The rollers 120 can be cylindrical or round like a dowel rod, but is not limited to being circular. Any shape that would allow a resistance band to be stretched and fed up and down toward the user about an axis to avoid friction incurred by the bands on the edge of the platform, such as an n-side polygonal shape (where n is a number) or irregular shapes. The rollers may alleviate the friction of any kind of material such as a rubber or latex resistance band, non-stretch or stretch strap, rope, or any material that one may use to pull something or provide resistance with.

Turning to FIG. 2, with reference to FIG. 1, the handle system 200 is shown. The handle system 200 may either grab, pull or support the resistance bands by one or more hook-shaped portion or hooking portion 201, carabiner, or any other compartment or mechanism which would be capable of holding onto one or multiple resistance bands. The handle system 200 has a gripping portion 202 where the user can grip all bands while not interfering with the band or multiple bands. The gripping portion 202 may extend between two or more hooking portions 201. For instance, the handle system 200 may comprise two hooking portions 201 located at distal and proximal ends of the handle system 200. The two hooking portions 201 may be generally parallel to each other. In another aspect, the grip portion 202 may extend between the hook-shaped portions 201. In some embodiments, a support 204 may additionally or alternatively extend between the hook-shaped portions 201 to provide support.

It is noted that the inner curvature 210 of the hook portion 201 may comprise a planar region 212 that may be generally planar, flat, or otherwise comprise a low curvature or angle. This may provide a surface in which a resistance band or bands may be disposed without causing the band to bend, twist, or experience unwanted tensile force. This may increase the life of the band. The planar region 212 may also become near-frictionless to the resistance bands supported through the addition of any type of bearing assembly, such as a rotating mechanism such (e.g., ball bearings, a rotating cylinder, etc.), to allow for even further tension equilibrium adjustments in the whole lifting system.

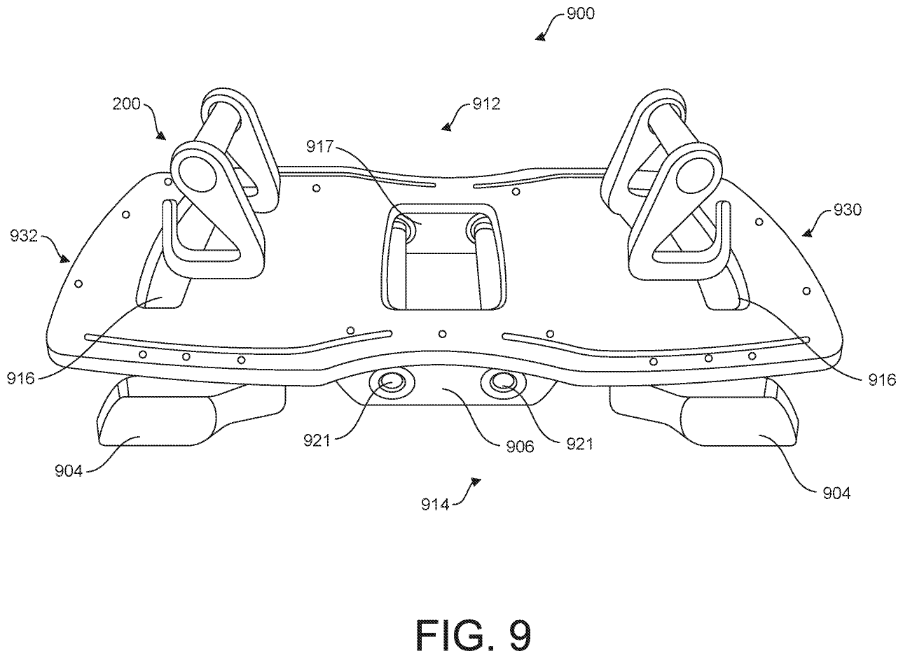

The grip portion 202 and the hook-shaped portion 201 may be configured such that the grip portion 202 and the planar region 212 of the hook-shaped portion 201 are biased to align with each other during operation, such as shown in FIG. 9. This allows resistance bands to rest within the appropriate place of the hook-shaped portion 201, away from the terminal end of the hook-shaped portion 201. As such, slippage or accidental release of the band is reduced or prevented during operation.

The platform 110 and the handle system 200 can comprise different materials, such as plastics, wood, metal, polyurethane, fiber-glass based, or any solid shape-maintaining material. In another aspect, the platform 110 and the handle system 200 can be manufactured via an appropriate technique for the materials, such as woodworking, injection molding, blow molding, any additive manufacturing processes such as 3D printing, or any subtractive manufacturing processes such as the use of a CNC machine. It is noted that various described components (e.g., platform 110, legs 104, handles 200) may comprise other or different shapes and sizes. The platform 110, for example, may be manufactured in different sizes as long as the user can safely and comfortably perform exercises.

In another aspect, a platform 110 can come in various shapes and assembly methods, as long as it is a rigid platform. A more compact or travel-friendly size could also be accomplished by the use of a collapsible platform via a hinge system or assembled by snapping or aligning two or more parts of the platform together or extending the width or length of the platform with some kind of a telescoping or sliding extension system. The platform 110 could also have a completely separate left and right side that are independent of themselves, not necessarily connecting. Each foot of the user would get its own independent platform with limited or no connection holding them together.

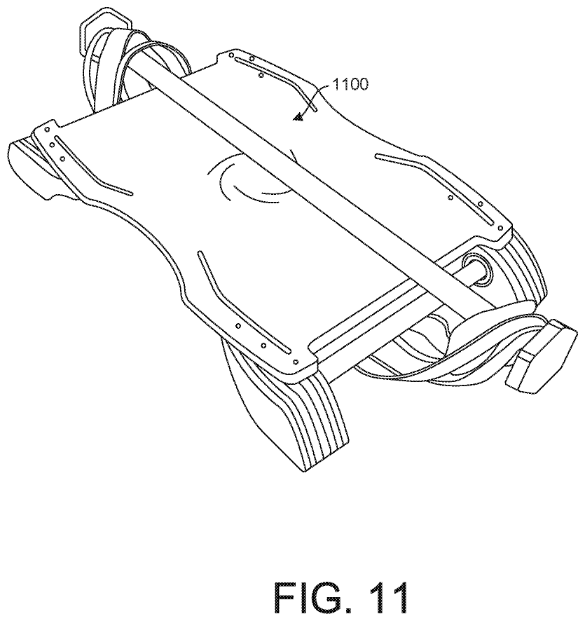

It is further noted that the system 100 may be utilized without handles, e.g., with resistance bands held by a user's hand, with gripping devices other than the handles 200, e.g., handle, straight bar, curl bar, chains, etc. An example of system 100 with a straight bar 1100 is shown in FIG. 11.

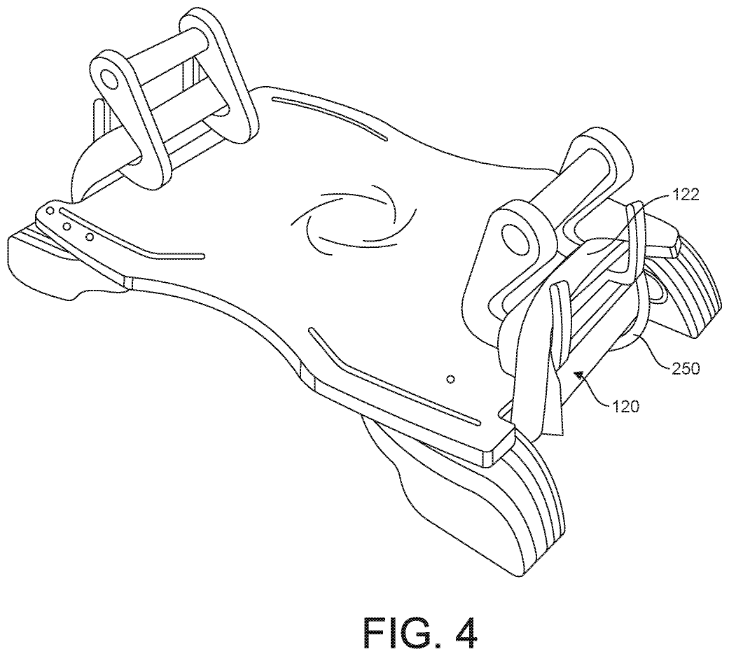

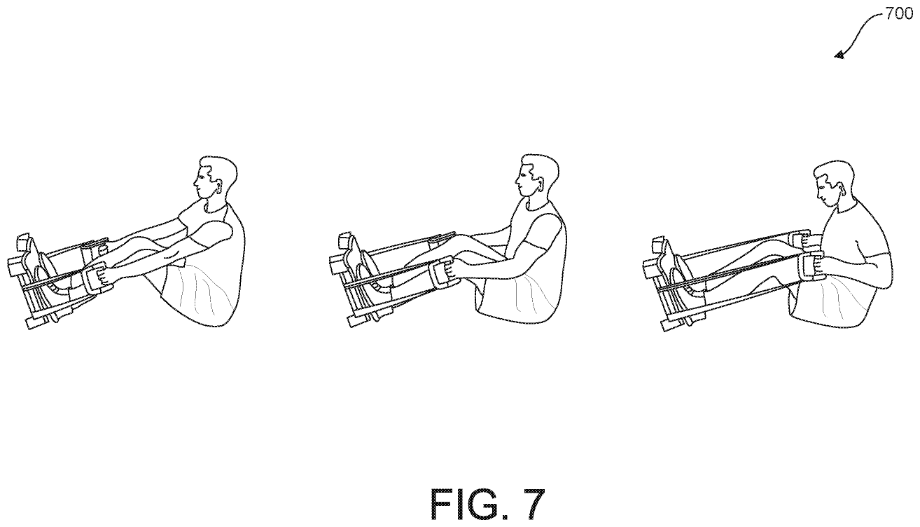

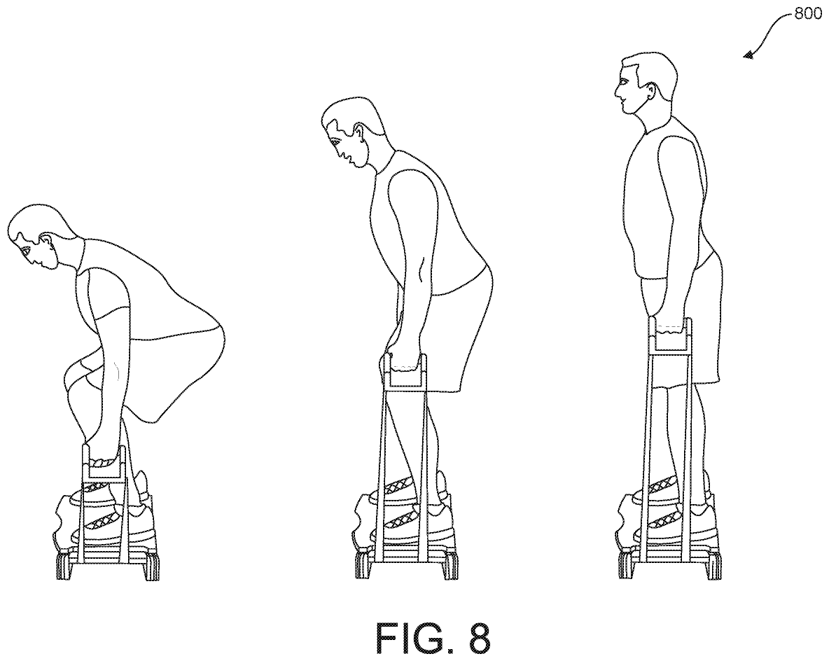

Turning to FIG. 4, there illustrated is the system 100 with two handles 200 as described herein. The handles 200 are illustrated with one or more resistive bands 250 attached thereto. The resistive bands 250 pass over the one or more rollers 120, wrap around the second side 114, and extend upwards towards the first side 112 at edges 130 and 132. This arrangement may allow for any number of exercises to be performed in different positions and targeting different muscle groups (e.g., upper body, lower body, etc.). For instance, a user may perform seated exercises (e.g., rows 700 as shown in FIG. 7), standing exercises (e.g., hex bar deadlift 800 as shown in FIG. 8), step back lunges, Romanian deadlift, single-leg Romanian deadlift, box deadlift, Bulgarian split squat, front squat, box single leg deadlift, overhead squats, seated rows, bent over rows, band bench (placing one's back on the platform and pushing out with arms), single-arm oblique raises for core strength (letting one of the handles stay anchored to the ground to hold one end of the resistance band, and only performing the lift with the other handle), bicep curls, triceps extensions, shoulder press, etc. It is noted that exercises may be performed geared towards maximizing resistance, maximizing cardio, or otherwise.

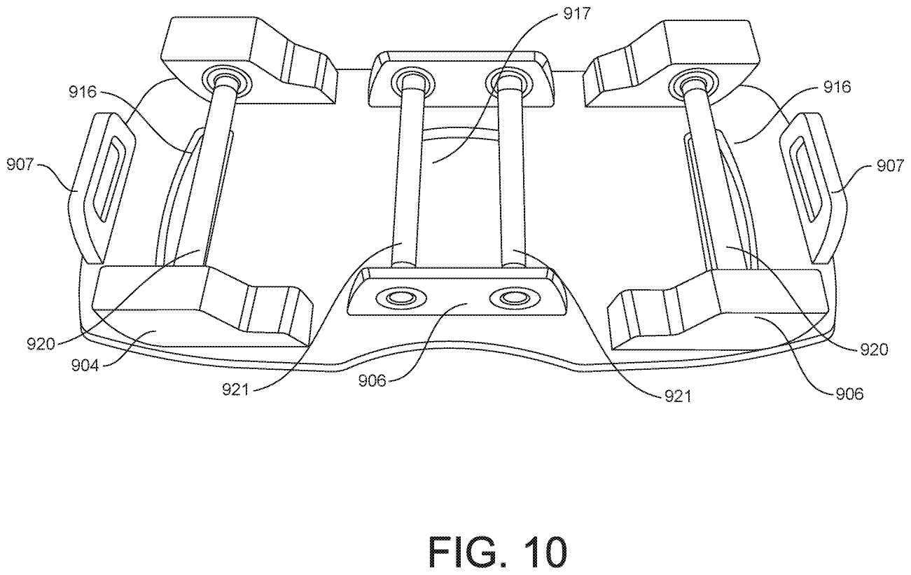

Referring now to FIGS. 9-10, there illustrated is a system 900 comprising a platform 910, at least one roller 920, and a plurality of handles 200. The platform 910 may comprise similar aspects to those described with reference to FIGS. 1-8. For instance, the platform 910 may comprise a first side 912 and a second side 914 opposed to the first side 912. The first side 912 may generally provide a surface on which a user may stand or otherwise apply pressure.

The second side 914 may comprise one or more supports 904, 906, 907 extending therefrom. The supports 904, 906, 907 may extend from the second side 914 for the same or different distances. As such, some or all of the supports 904, 906, 907 may contact a surface, e.g., a floor. As shown, supports 904 and 907 may contact a floor when oriented with the second side 914 directed towards the floor. The supports 906 are illustrated as not touching the floor. In various embodiments, supports 906 may extend to the floor. Moreover, supports 907 may be sized and shaped to allow a user to grasp the supports 907 to utilize as a handle for carrying the system 900.

Supports 906 and 904 may respectively comprise one or more rollers 921, 920 disposed there between. Moreover, the position of the one or more rollers 921, 920 may generally be aligned with cutouts 917, 916 such that resistance bands may pass around the one or more rollers 921, 920 and extend to a user on the first side 912. As depicted, the supports 906 may be located generally closer to a center of the platform 910 than the supports 904. The placement of the supports 906, the cutout 917 and the rollers 921 allow a user to select the rollers 921 for some exercises requiring a closer distance between ends of a resistance band, while the placement of the one or more rollers 920, cutouts 916, and supports 904 allow a user to select the rollers 920 for some exercises requiring a further distance between ends of a resistance band. It is further noted that the one or more rollers 917 may be planar with each other (e.g., are disposed the same distance from the second surface 914), and non-planar with the one or more rollers 920 (e.g., are disposed a different distance from the second surface 914 than the supports 920. In an aspect, this may allow the rollers 917 to not contact a resistance band passing over the rollers 920.

Moreover, as depicted, the cutouts 916 may be located a distance from first edge 930 and second edge 932. For instance, the cutouts 916 may be framed on all sides. It is noted that the platform 910 may include other or different cutouts, rollers, and supports as described herein.

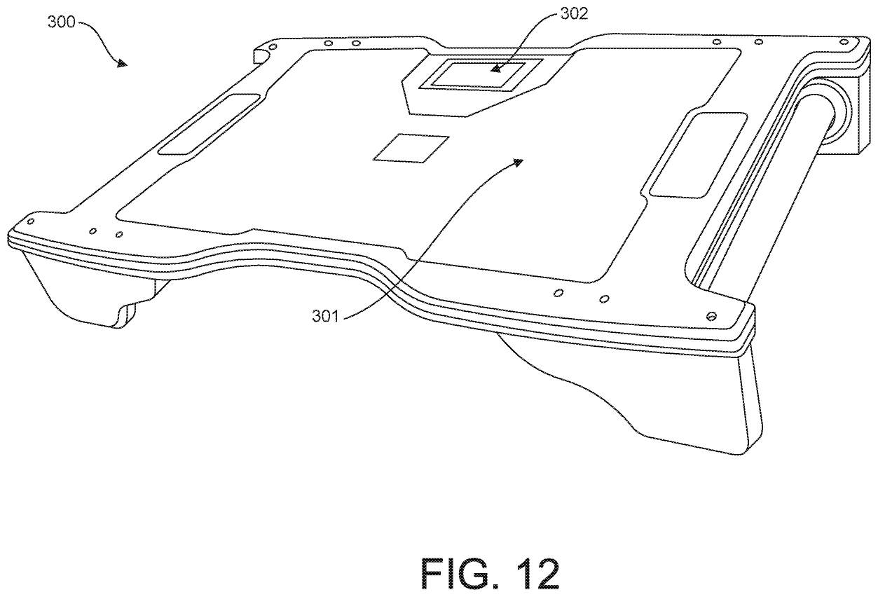

As shown in FIG. 12, the device or system may be a “smart platform” 1200, where there is a way of telling the user specifically how much resistance is created by the resistance bands lifted with continuous real time feedback on the amount of resistance. The components include an embedded measuring platform 1201, such as a scale, and the resistance experienced by the user can be viewed in the front of the platform via an alphanumeric screen 1202. This may be particularly useful to users who are inexperienced with resistance bands and desire a weight measurement in traditional units such as pounds or kilograms.

In FIG. 12 As the “smart platform” 1200 could be connected to sensors that would give both the resistance lifted and acceleration to the top of a lift, an instructor could remotely monitor and advise on the performance of a user when connected through the internet. An application that would connect to a screen could show both the user and the instructor the rate of change in the resistance, thus giving explicit data whether the user is tiring out or needs to reduce or increase resistance. The rollers on either side of the platform or the measuring scale embedded into the device's platform would be able to measure the velocity and acceleration of the lift performed and be able to tell the user how they stack up to other athletes or lifters. Based off of the various acceleration and resistance outputs a user could tell or be instructed remotely what part of their athleticism they are training (e.g. power output vs. absolute strength). Whether a user is quick with light loads or heavy loads, this measuring system will be able to tell them and advise according to their goals.

An instructor could monitor one or multiple remote users from across the globe and give individual and personalized feedback on the resistance they would be using as well as the technique utilized by the user through a one or two-way camera. An instructor may not be necessary as the communication and advice-giving could be replaced by certain algorithms or formulas that advise the lifter to adjust their lifting resistance, pace, or form as they cross certain ranges or thresholds.

In another aspect, the smart platform 1200 may communicate with various user devices such as smartphones, laptop computers, wearable devices (e.g., fitness monitors), set-top boxes, or the like. Fitness activity and exercises may be communicated to the user device to track progress, exercises, monitor goals, or the like.

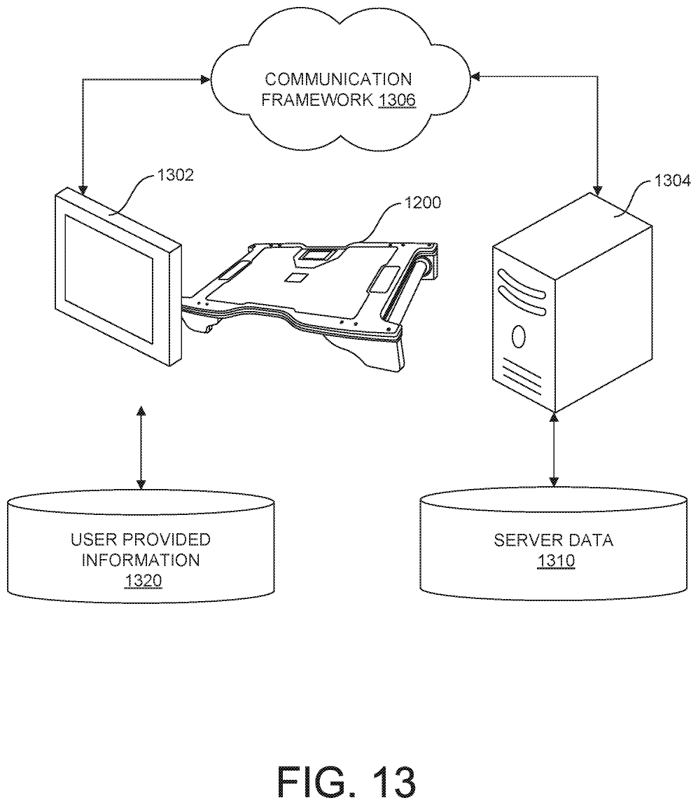

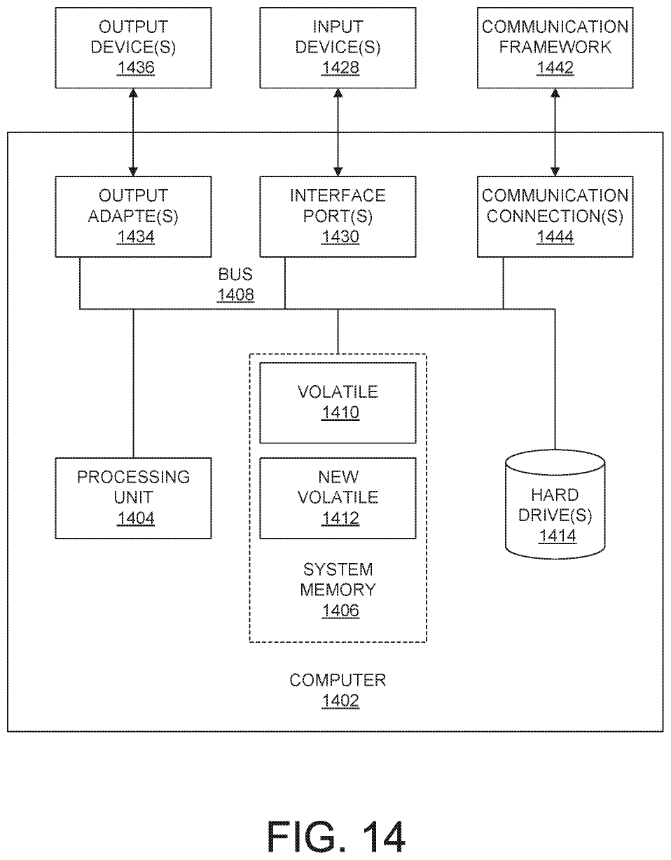

What has been described above may be further understood with reference to the following figures. FIGS. 13 and 14 provide exemplary operating environments or systems capable of implementing one or more systems, apparatuses, or processes described above. FIGS. 13 and 14 are not intended to limit the scope of such systems, apparatuses, or processes. By way of example, computing environment 1300 may refer to one or more embodiment of the various embodiments described with reference to the above figures. However, variations to computing environment 1300 may be obvious to achieve aspects or processes described herein.

FIG. 13 is a schematic diagram of a computing environment 1300 in accordance with various disclosed aspects. It is noted that computing environment 1300 may include various other components or aspects. As depicted, computing environment 1300 may include one or more client(s) 1302, one or more server(s) 1304, one or more client data store(s) 1320, one or more server data store(s) 1310, and a communication framework 1306.

While depicted as a desktop computer(s), client(s) 1302 may include various other devices that may comprise hardware and/or software (e.g., program threads, processes, computer processors, non-transitory memory devices, etc.). In an example, client(s) 1302 may include a smart platform 1200, laptop computers, smart phones, tablet computers, wearables, etc. The client(s) 1302 may include or employ various aspects disclosed herein. For example, client(s) 1302 may include or employ all or part of various systems and processes disclosed herein.

Likewise, server(s) 1304 may include various devices that may comprise hardware and/or software (e.g., program threads, processes, computer processors, non-transitory memory devices, etc.). Server(s) 1304 may include or employ various aspects disclosed herein. For example, server(s) 1304 may include or employ all or part of various systems and processes disclosed herein. It is noted that server(s) 1304 and client(s) 1302 may communicate via communication framework 1306. In an exemplary communication, client(s) 1302 and server(s) 1304 may utilize packeted data (e.g., data packets) adapted to be transmitted between two or more computers. For instance, data packets may include coded information associated with printing requests, orders, available printers, parameters associated with print-receptive media, or the like.

Communication framework 1306 may comprise various network devices (e.g., access points, routers, base stations, etc.) that may facilitate communication between client(s) 1302 and server(s) 1304. It is noted that various forms of communications may be utilized, such as wired (e.g., optical fiber, twisted copper wire, etc.) and/or wireless (e.g., cellular, Wi-Fi, NFC, etc.) communications.

In various embodiments, client(s) 1302 and server(s) 1304 may respectively include or communicate with one or more client data store(s) 1320 or one or more server data store(s) 1310. The data stores may store data local to client(s) 1302 or server(s) 1304.

In at least one embodiment, a client of client(s) 1302 may transfer data describing a print order, user account data, printer device data, or the like to a server of server(s) 1304. The server may store the data and/or employ processes to alter the data. For example, the server may transmit the data to other clients of client(s) 1302.

FIG. 14 is a block diagram of a computer system 1400 that may be employed to execute various disclosed embodiments. It is noted that various components may be implemented in combination with computer-executable instructions, hardware devices, and/or combinations of hardware and software devices that may be performed by computer system 1400.

Computer system 1400 may include various components, hardware devices, software, software in execution, and the like. In embodiments, computer system 1400 may include computer 1402. Computer 1402 may include a system bus 1408 that couples various system components. Such components may include a processing unit(s) 1404, system memory device(s) 1406, disk storage device(s) 1414, sensor(s) 1435, output adapter(s) 1434, interface port(s) 1430, and communication connection(s) 1444. One or more of the various components may be employed to perform aspects or embodiments disclosed herein. In an aspect, the computer system 1400 may “learn,” such as described above user preferences based upon modifications of workout routines by users, through the rating of workout routine both positively and negatively. For example, the computer system 1400 may modify a particular workout routine (or a set thereof) as the majority of users or supermajority thereof have disapproved of the workout routine (such as for strength, speed, cardiovascular challenge, length of time, or a variety of these factors). The computer system 1400 may dynamically push out the revised workout routine or receive the revised workout routine as applicable.

The processing unit(s) 1404 may comprise various hardware processing devices, such as single-core or multi-core processing devices. Moreover, processing unit(s) 1404 may refer to a “processor,” “controller,” “computing processing unit (CPU),” or the like. Such terms generally relate to a hardware device. Additionally, processing unit(s) 1404 may include an integrated circuit, an application specific integrated circuit (ASIC), a digital signal processor (DSP), a field-programmable gate array (FPGA), a programmable logic controller (PLC), a complex programmable logic device (CPLD), a discrete gate or transistor logic, discrete hardware components, or the like.

System memory 1406 may include one or more types of memory, such volatile memory 1410 (e.g., random access memory (RAM)) and non-volatile memory 1412 (e.g., read-only memory (ROM)). ROM may include erasable programmable ROM (EPROM), electrically erasable programmable ROM (EEPROM). In various embodiments, processing unit(s) 1404 may execute computer executable instructions stored in system memory 1406, such as operating system instructions and the like.

Computer 1402 may also include one or more hard drive(s) 1414 (e.g., EIDE, SATA). While hard drive(s) 1414 are depicted as internal to computer 1402, it is noted that hard drive(s) 1414 may be external and/or coupled to computer 1402 via remote connections. Moreover, input port(s) 1430 may include interfaces for coupling to input device(s) 1428, such as disk drives. Disk drives may include components configured to receive, read and/or write to various types of memory devices, such as magnetic disks, optical disks (e.g., compact disks and/or other optical media), flash memory, zip drives, magnetic tapes, and the like.

It is noted that hard drive(s) 1414 and/or other disk drives (or non-transitory memory devices in general) may store data and/or computer-executable instructions according to various described embodiments. Such memory devices may also include computer-executable instructions associated with various other programs or modules. For instance, hard drives(s) 1414 may include operating system modules, application program modules, and the like. Moreover, aspects disclosed herein are not limited to a particular operating system, such as a commercially available operating system.

Input device(s) 1428 may also include various user interface devices or other input devices, such as sensors (e.g., microphones, pressure sensors, light sensors, etc.), scales, cameras, scanners, facsimile machines, and the like. A user interface device may generate instructions associated with user commands. Such instructions may be received by computer 1402. Examples of such interface devices include a keyboard, mouse (e.g., pointing device), joystick, remote controller, gaming controller, touch screen, stylus, and the like. Input port(s) 1430 may provide connections for the input device(s) 1428, such as via universal serial ports USB ports), infrared (IR) sensors, serial ports, parallel ports, wireless connections, specialized ports, and the like.

Output adapter(s) 1434 may include various devices and/or programs that interface with output device(s) 1436. Such output device(s) 1436 may include LEDs, computer monitors, touch screens, televisions, projectors, audio devices, printing devices, or the like.

In embodiments, computer 1402 may be utilized as a client and/or a server device. As such, computer 1402 may include communication connection(s) 1444 for connecting to a communication framework 1442. Communication connection(s) 1444 may include devices or components capable of connecting to a network. For instance, communication connection(s) 1444 may include cellular antennas, wireless antennas, wired connections, and the like. Such communication connection(s) 1444 may connect to networks via communication framework 1442. The networks may include wide area networks, local area networks, facility or enterprise-wide networks (e.g., intranet), global networks (e.g., Internet), satellite networks, and the like. Some examples of wireless networks include Wi-Fi, Wi-Fi direct, BLUETOOTH™, Zigbee, and other 802.XX wireless technologies. It is noted that communication framework 1442 may include multiple networks connected together. For instance, a Wi-Fi network may be connected to a wired Ethernet network.

The terms “component,” “module,” “system,” “interface,” “platform,” “service,” “framework,” “connector,” “controller,” or the like are generally intended to refer to a computer-related entity. Such terms may refer to at least one of hardware, software, or software in execution. For example, a component may include a computer-process running on a processor, a processor, a device, a process, a computer thread, or the like. In another aspect, such terms may include both an application running on a processor and a processor. Moreover, such terms may be localized to one computer and/or may be distributed across multiple computers.

What has been described above includes examples of the present specification. It is, of course, not possible to describe every conceivable combination of components or methodologies for purposes of describing the present specification, but one of ordinary skill in the art may recognize that many further combinations and permutations of the present specification are possible. Each of the components described above may be combined or added together in any permutation to define embodiments disclosed herein. Accordingly, the present specification is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An exercise assembly comprising:
a platform comprising a first side and a second side, the first side operatively bearing a user;
at least one roller device disposed beneath the second side when viewed from the first side; and
at least one cutout formed through the platform from the first side to the second side, the at least one cutout operatively disposed to provide line of sight with the at least one roller when viewed from the first side, such that a resistive band may pass through the at least one cutout and pass over the at least one roller device, and wherein terminal ends of the resistive band extend above the first side.

2. The exercise assembly of claim 1, further comprising at least one support extending from the second said.

3. The exercise assembly of claim 2, wherein the at least one roller is disposed between two of the at least one support.

4. The exercise assembly of claim 3, wherein the platform comprises a first edge and a second edge and wherein the at least one support comprises four supports disposed at general corners of the second side of the platform, and wherein the at least one roller comprise a first roller disposed between two of the supports proximal the first edge and a second roller disposed between the two other supports proximal the second edge.

5. The exercise assembly of claim 1, wherein the at least one roller comprises at least one of a dowel rod, an axle comprising one or more bearings, an axle mounted with or on bearings, an axle with one or more bearing beads circumventing the axle, or ball bearing surfaces.

6. The exercise assembly of claim 1, wherein the platform comprises a first edge and a second edge opposite the first edge.

7. The exercise assembly of claim 6, wherein the one or more cutouts comprise a first cut out proximal the first edge and a second cutout proximal the second edge.

8. The exercise assembly of claim 6, wherein the one or more cutouts comprise a first cut out and a second cutout wherein the first cutout is positioned a distance from the first edge and the second cutout is positioned the distance from the second edge.

9. The exercise assembly of claim 6, wherein the one or more cutouts comprise a cutout positioned in the general center of the platform.

10. The exercise assembly of claim 1, further comprising an anti-slip surface disposed on the first side.

11. A exercise assembly comprising:
a platform comprising a first side and a second side, a first edge, and a second edge, the first side operatively bearing a user, the second side opposed to the first side, and the first edge opposed to the second edge;
a plurality of supports extending from the second side;
at least one roller device disposed beneath the second side when viewed from the first side, and rotatably attached to the plurality of supports; and
at least one cutout formed through the platform from the first side to the second side, the at least one cutout operatively disposed to provide line of sight with the at least one roller when viewed from the first side, such that a resistive band may pass through the at least one cutout and pass over the at least one roller device, and wherein terminal ends of the resistive band extend above the first side.

12. The exercise assembly of claim 10 further comprising at least one handle operatively attaching to terminal ends of the resistive band from the first side.

13. The exercise assembly of claim 11, wherein forcing the at least one handle away from the first side operatively stretches the resistive bands, and wherein the at least one roller device operatively allows the resistive band to deform such that resistance on terminal ends of the resistive bands are generally equalized.

14. The exercise assembly of claim 11, wherein the at least one handle comprises a gripping portion operatively providing a gripping surface for a user and at least one hooking portion operatively attaching to the terminal ends of the resistive band.

15. The exercise assembly of claim 13, wherein the at least one hooking portion further comprises a planar surface for receiving the resistive band.

16. The exercise assembly of claim 13, wherein the at least one hooking portion further comprises at least one roller device for receiving the resistive band.

17. The exercise assembly of claim 11, wherein the gripping portion comprises an ergonomic handle.

18. An exercise assembly, comprising:
a platform comprising a first side and a second side, a first edge, and a second edge, the first side operatively bearing a user, the second side opposed to the first side, and the first edge opposed to the second edge, and further comprising at least one sensor disposed on or within the platform for sensing pressure operatively applied by a user during exercise;
a plurality of supports extending from the second side;
at least one roller device disposed beneath the second side when viewed from the first side, and rotatably attached to the plurality of supports; and
at least one cutout formed through the platform from the first side to the second side, the at least one cutout operatively disposed to provide line of sight with the at least one roller when viewed from the first side, such that a resistive band may pass through the at least one cutout and pass over the at least one roller device, and wherein terminal ends of the resistive band extend above the first side.

19. The exercise assembly of claim 17, wherein the at least one sensor is operatively coupled to a controller that determines an amount of resistance during an exercise.

20. The method of claim 18, wherein the controller further determines a balance metric associated with the user during the exercise.
